(19) United States
(12) Patent Application Publication
(10) Pub. No.: US 2018/0369446 A1
(43) Pub. Date: Dec. 27, 2018

(54) CURABLE SILICONE (PRE-) POLYMER COMPOSITION COMPRISING A CONTRAST AGENT

(71) Applicant: CAR Holding B.V., Maastricht (NL)

(72) Inventors: Alexander Cornelis de Vries, Maastricht (NL); Henri Lorenzo Frederik Brom, Maastricht (NL)

(21) Appl. No.: 15/777,066

(22) PCT Filed: Nov. 18, 2016
(86) PCT No.: PCT/NL2016/050808

§ 371 Date: May 17, 2018

(30) Foreign Application Priority Data
Nov. 18, 2015 (NL) 2015807
Nov. 18, 2015 (NL) 2015809

(51) Int. Cl. A61L 24/00; A61L 31/12; A61L 31/18

(52) U.S. Cl. CPC A61L 24/0089; A61L 31/128; A61L 2400/06; A61L 2430/36; A61L 31/18

(57) ABSTRACT

The invention relates to a kit of parts suitable for preparing a cured biocompatible silicone polymer material including an X-ray contrast agent, the kit comprising two or more containers containing a fluid component, which components—when mixed—form a fluid curable biocompatible polymer composition, which upon curing forms the cured biocompatible silicone polymer material, wherein a first container contains a fluid component A, which component A comprises a curable silicone (pre-)polymer and a curing agent and which component A is essentially free of curing catalyst and metallic X-ray contrast agent particles, and

a second container contains a fluid component B, which component B is a dispersion comprising metallic X-ray contrast agent particles and which is essentially free of curing agent.

The invention relates to a kit of parts for providing a biocompatible polymer composition that is suitable for use in a medical treatment, in particular in the treatment of a subject having an aneurism.

Treatments of aneurisms with curable polymer compositions have been a topic of research and development for several decades.

Aneurysms are local dilatations in blood vessels, in particular arteries that gradually enlarge in time. Unless an aneurysm is adequately treated, it may eventually rupture and cause severe damage to the body, possibly even result in shock or death. Aortic aneurysms are in particular an important cause of death in human adults of 55 years and older.

Traditional repair of an aneurysm entails a major operation with an incision into the aneurysm, evacuation of the clot that is usually contained within, placement of a synthetic graft and wrapping of the graft with the remnants of the artery wall.

A more recent development is the endovascular stent technique, that has become a common technique to treat abdominal aortic aneurisms. This procedure does not require general anaesthesia and can be done less invasively by simply placing a self-expanding stent via a catheter passed through one of the femoral arteries into the aneurysm to stabilise it. Less fit patients are able to withstand the procedure, hospital stay is cut to 1 to 2 days, and post-operative recovery is shortened considerably.

In WO 95/08289 it is proposed to repair cardiovascular anomalies via the introduction of a photo-activatable biopolymer, which is introduced to the anomaly via a catheter system, after which the polymer is cross-linked. The publication mentions several examples of potentially suitable polymers, wherein it is suggested to be advantageous that the polymers are not only photo-activatable but also biodegradable and resorbable.

A catheter system for delivering fluid materials, such as medicaments to a body vessel is reported in EP-A 0 667 131. The fluid material is for example a mixture comprising an epoxy resin that cures in the presence of ions.

EP-A 1 435 249 relates to biocompatible polymer composition for use in the treatment of in vivo vessel repair, such as the repair of an aortic aneurism, with satisfactory properties with respect to—amongst others—curing behaviour, low toxicity, biocompatibility and durability in vivo.

The inventors found though, that, in case the curable polymer is based on a silicone pre-polymer it is problematic to provide a sufficiently storage-stable curable polymer composition, containing a contrast fluid (providing radio opacity), like Omnipaque®, or a metallic contrast agent, like tantalum.

It is noted that tantalum is known in as a radio opaque material in an ethylene vinyl alcohol (EVOFA) polymer composition, namely Onyx®. When used at 33% w/v of tantalum powder in formulation yields good visualization of Onyx during embolization procedures. The product functions in a fundamentally different way than an in situ curable (pre-)polymer composition. Onyx® is mixed with a powder comprising tantalum shortly before administration into a blood vessel and precipitates in-situ, whereby the particles comprising tantalum are trapped and encapsulated within the EVA polymer. However, when such powder was added to a fluid silicone (pre-) polymer composition, no satisfactory results were obtained. If added to the curable silicone (pre-)polymer composition, dispersion problems, processing problems and lack of storage stability were observed. Storage stability (no unacceptable changes in viscosity, no unacceptable settling of particles) is desired because it allows the provision of a ready-to-use/easy-to-administer product to surgeons. The inventors found that undesired polymerisation reactions (prior to intended use) are a cause of this. The use of a metallic contrast agent has in particularly be found a challenge in a curable silicone composition intended for use in the treatment of a blood vessel wherein the blood vessel should remain patent after the composition has been administered and cured, and wherein time needed to apply the composition and the curing time is a critical factor, as is the case if the compositions is administered to the aorta, in particular when used in the treatment of an aortic aneurism.

It thus remains a challenge to provide a curable silicone-based composition for use in a medical treatment, such as in the treatment of a blood vessel, in particular of an aortic aneurism, whilst the composition has easy handling properties, including radio-opacity, and a satisfactory storage stability.

It is an object of the present invention to provide an alternative to known polymer compositions, containing a contrast agent, for use in the treatment of a subject having an aneurism, in particular an alternative that allows sufficiently stable storage prior to the intended use, yet is relatively easy to handle, has a satisfactory application time, and a satisfactory curing time, also in case the composition is for use in a treatment in the aorta of a subject.

It has now been found that this object is met, by providing a specific combination of a curable silicone (pre-)polymer and a specific X-ray contrast agent.

Accordingly, the invention relates to a kit of parts suitable for preparing a cured biocompatible silicone polymer material including a metallic X-ray contrast agent, the kit comprising two or more containers containing a fluid component, which components—when mixed—form a curable biocompatible polymer composition, which upon curing forms the cured biocompatible silicone polymer material, wherein

a first container contains a fluid component A, which component A comprises a curable silicone (pre-)polymer and a curing agent, and a second container contains a fluid component B, which component B is a dispersion comprising metallic X-ray contrast agent particles.
The kit of parts generally further comprises a curing catalyst, preferably a platinum complex, which curing catalyst is present in component B or in a third container, comprising a fluid component C, although in principle it is also possible to cause curing with radiation, e.g. electromagnetic radiation, such as UV-curing. Component A is generally essentially free of catalysts, catalyzing the curing at ambient temperature (about 25 C).
Component B is generally essentially free of curing agents, participating in crosslinking of the curable silicone (pre-)polymer.

It has been found that a kit of parts according to the invention is storage-stable, i.e. it remains sufficiently stable in terms of a lack of unacceptable reactions prior to intended use, lack of unacceptable settling of the contrast agent particles and no undesired changes in flowing properties, for a sufficient time between production, e.g. in a factory, and its use, e.g. in a clinic or hospital. Usually, the kit of parts can be stored at ambient temperature for more than two months, without unacceptable settling. In particular, the kit of parts can be stored for about 6 months or more, e.g. 1-3 years, preferably at ambient temperature or in cooled (e.g. at about 4 C). This is achievable in accordance with the invention, without needing a curing-inhibitor.

The invention further relates to a method for preparing a curable silicone polymer composition, comprising mixing the components A and B, or—if component C is present—the components A, B and C of a kit of parts according to the invention.

The invention further relates to a curable silicone polymer composition, obtainable by a method according to the invention.

The invention further relates to a method for preparing a cured biocompatible silicone polymer material including an X-ray contrast agent, comprising curing the composition according to the invention. Such method may also be carried out in a non-medical setting.

The invention further relates to a cured material, obtainable by the method for preparing a cured material according to the invention.

The kit of parts or curable polymer composition according to the invention may be used for various purposes.

In particular the kit of parts or curable polymer composition is useful in the treatment of a subject having a vascular disease.

In an advantageous embodiment, the treatment of the vascular disease involves the treatment of a human or another mammal, wherein in situ a stent of the cured silicone polymer material is formed, using the kit of parts or curable polymer composition of the invention.

In an advantageous embodiment a use in accordance with the invention comprises the treatment of an aortic aneurism, preferably an abdominal aortic aneurism, a thoracic aortic aneurism or an aneurism in an ileac artery. In a specific embodiment, the treatment comprises a repair of an endoleak of a graft or stent-graft in an artery, in particular a type II or a type I endoleak.

It is also possible to use the kit of parts or curable polymer composition according to the invention in the treatment of an aneurism, wherein the composition is for use as an adjuvant filling of the aneurism in a method wherein an endo-graft is placed in the aneurism.

Besides treatment of a vascular disease, a kit of parts or curable polymer composition according to the invention is also particularly suitable for use in a prophylactic treatment of a bone, preferably a hip or a collarbone.

The term “or” as used herein means “and/or” unless specified otherwise.

The term “a” or “an” as used herein means “at least one” unless specified otherwise.

When referring to a “noun” (e.g. a compound, an additive etc.) in singular, the plural is meant to be included, unless specified otherwise.

The term “substantial(ly)” or “essential(ly)” is generally used herein to indicate that it has the general character or function of that which is specified, for instance when referring to essentially spherical it means that it has at least the general appearance of a sphere. When referring to a quantifiable feature, these terms are in particular used to indicate that it is for more than 50%, in particular at least 75%, more in particular at least 90%, even more in particular at least 95% of the maximum that feature.

The term ‘ essentially free’ is used herein to indicate that a substance is not present (below detection limit using standard analytical technology) or present in such a low concentration, e.g. less than 0.1% or less than 0.01%, that is does not significantly affect a function of a product in which it is present and/or that there is no requirement to label the presence of the substance, in particular in the USA or the European Union.

When referring to an amount-related feature, the amount in terms of weight is meant, unless specified otherwise.

When using the term ‘about’ this in particular signifies a deviation of 10% or less, more in particular of up to 5%, more in particular of up to 3%, more in particular up to 2% from the indicated value.

For the purpose of clarity and a concise description, features are described herein as part of the same or separate embodiments, however, it will be appreciated that the scope of the invention may include embodiments having combinations of all or some of the features described.

The kit of parts can simply be used to prepare the curable fluid polymer composition by mixing the components. This is conveniently achieved using a static mixer, although another type of mixer can be used. The volume to volume ratio of component A:component B is usually in the range of 1:3 to 3:1, preferably about 1:1. If component C is present, the ratio component B:C is usually about 1:3 to 3:1, preferably about 1:1. Good results have been achieved though with a two-component system. The containers for the fluid components preferably are part of a multi-barrel syringe (e.g. double barrel or triple barrel), each barrel containing one of the fluid components. In particular for such kit of parts, it is convenient that about equal volumes of each of the components are present in the kit. Dependent on the intended use, the kit may further comprise one or more additional items, in particular one or more items selected from the group consisting of static mixers, catheters, catheter balloons, stents and endo-grafts. Further, the kit may be provided with instructions for use.

Substances for the components of the kit of parts can generally be based on EP-A 1 435 249, of which the contents are incorporated by reference. The components are usually essentially free of curing inhibitors. In addition to the substances described in EP-A 1 435 249, a metallic contrast agent is present in Component B, Further, one or more of the components A and B (and if present, C), may comprise a silicone base.

One or more of the components are preferably free of components dissolvable in water or blood. Preferably, at least Component A, Component B, and—if present—Component C are free of components dissolvable in water or blood. The polymer composition obtained by mixing the components of the kit of parts is curable without the necessity of any component dissolving or diffusing out of said composition. In particular, the components of the kit of parts, at least Component A and B and—if present—Component C, are preferably free of solvents, like DMSO, that are soluble in water and/or water-soluble buffer components, of which glycylglycine and HEPES are examples.

Before curing the composition is fluid, i.e. it is capable of flowing (usually while being pumped) through a lumen for delivery of the composition into the aneurism.

The substances are preferably chosen to provide fluid components having a viscosity in the range of 2 000 to 12 000 cSt (corresponding to 2-12 Pa·s for a composition with a density of 1 000 kg/m³) at 25° C., preferably in the range of 3 000 to 10 000 cSt, more preferably in the range of 4 000 to 8 000 cSt. The components may have the same or a different viscosity, the difference in viscosity generally being less than 2 000 cSt, preferably being 0-1 500 cSt. The mixture of the components (the curable biocompatible polymer composition) preferably has a viscosity in the range of 2 000 to 12 000 cSt at 25° C., preferably in the range of 3 000 to 10 000 cSt, more preferably in the range of 4 000 to 8 000 cSt.

In a specific embodiment, the viscosity of the mixture of the components (that form the curable biocompatible polymer composition) is higher than 12 000 cSt, in particular at least 15 000 cSt, more in particular at least 20 000 cSt. Such a high viscosity would e.g. be considered to further reduce curing time, or in a situation wherein it is considered that at a lower viscosity may not fully satisfactorily reduce the risk of down-stream embolization. For administering a curable composition with a higher viscosity a more powerful pumping device may be needed. In view thereof, the viscosity will typically be less than 40 000 cSt, in particular less than 30 000 cSt, more in particular less than 25 000 cSt.

In a specific embodiment, the kit of parts respectively the biocompatible curable polymer composition is for use in a method for treating an aneurysm, as described in e.g. U.S. Pat. No. 7,530,988 or U.S. Pat. No. 8,048,145. Herein aneurysms are treated by filling a double-walled filling structure with a curable filling medium (such as a biocompatible curable polymer composition). The method comprises in particular: positioning at least one double-walled filling structure across the aneurysm; filling at least one filling structure with the biocompatible curable polymer composition so that an outer wall conforms to the inside of the aneurysm and an inner wall forms a generally tubular lumen to provide for blood flow; supporting the tubular lumen while and/or after the filing structure is being filled; hardening the biocompatible curable polymer composition while the tubular lumen remains supported; and

removing support after the filling material has hardened.
In such embodiment, the viscosity of the mixture of components (that form the curable biocompatible polymer composition) is usually lower than 2000 cSt, e.g. about 1800 cSt or less, in particular 1600 cSt or less. Such composition is in particular suitable in an embodiment wherein the mixture is administered to the body of a subject to be treated without the composition being in direct contact with the blood flow at least not until it has sufficiently cured. The viscosity of such mixture could be as low as 100 cSt or even lower. Preferably, the viscosity of such mixture is at least 500 cSt, more preferably at least 1000 cSt.

The viscosity as defined herein is the kinematic viscosity in cSt as measured by Brookfield viscosimeter (UK), model ND J-1 and/or rheometer RMS 800 from Rheometrics, USA. The kinematic viscosity of a fluid in cSt corresponds to the dynamic viscosity in mPa·s divided by the density of the fluid in g/cm³.

The mixture of the components (the curable biocompatible polymer composition) is curable in the presence of a curing catalyst at 25° C. to form a cured material. The material preferably has an elongation until rupture of at least 5%, in particular of 25-500%, more in particular of 50-250%. The elastic modulus of the cured material at 25° C. preferably is at least 1 MPa, in particular 2-40 MPa, more in particular 3-20 MPa. Preferably, after curing of the composition the resulting material has a stress value of at least 5 kPa at 1% strain, more preferably of at least 30 kPa at 20% strain, even more preferably a stress value of at least 1 MPa at 50% strain (As determined with Zwick 1445 or with DMA 7, Perkin-Elmer). The glass transition temperature (Tg) of a cured material obtained from a composition according to the invention is typically less than 37° C. Preferably the Tg is less than 25° C. Very good results have been achieved with a material having a Tg of less than −25° C. (Tg is the value as measured by differential scanning calorimetry (DSC) on a DSC 7, Perkin-Elmer.

The elongation until rupture is defined herein is the value as measured by a Zwick 1445 tensile strength tester (Germany).

The elastic modulus as defined herein is the value as measured by dynamic mechanical analyser, DMA 7 from Perkin-Elmer (USA).

The silicone (pre-)polymer comprises a curable monomer, oligomer or a curable polymer. Before curing the (pre-)polymer typically comprises one or more functional groups that allow further polymerisation, e.g. by cross-linking, to form the matrix of the composition after it has been cured. Particularly good results have been achieved with a vinyl-terminated silicone polymer.

The number of monomeric units or molecular weight of the matrix pre-polymer is not particularly critical, as long as it provides a suitable viscosity in the composition. Good results have been obtained with a matrix pre-polymer having at least 3, preferably at least 5, more preferably at least 20 monomeric units, before curing is initiated. For practical reasons, the matrix-polymer generally comprises less than 20 000 monomeric units, preferably less than 1 000, more preferably less than 100, in particular 50 or less, more in particular 10 or less before curing is initiated.

The number average molecular weight of the silicone pre-polymer may for example be in the range of 500 to 400 000 gram/mol, in particular in the range of 6 000 to 280 000 gram/mol.

The amount of silicone (pre-)polymer can be chosen within wide limits, depending upon the desired viscosity and other properties and may be adequately determined by the skilled professional on the basis of the present disclosure and references cited herein. Preferably the concentration of the silicone pre-polymer is 10 to 85 wt. % based on the total weight of the composition, more preferably in the range of 25-80 wt. %, in particular in the range of 50 to 75 wt. %, more in particular in the range of 60 to 70 wt. %.

Preferably a silicon (pre-)polymer used in a composition according to the invention has a start viscosity (i.e. before mixing it to a composition according to the invention) of at least 300 cSt. More preferably the start viscosity is in the range of 300 to 1 500 cSt.

Highly preferred is a polydialkylsiloxane polymer, comprising at least two vinyl groups, preferably at the terminal ends. In a specific embodiment, the polydialkylsiloxane polymer has (on average) 3-5 vinyl groups per molecule.

Very good results have been achieved with a vinyldimethylsiloxy terminated polydimethylsiloxane (PDMS). A highly preferred polydimethylsiloxane polymer is shown in formula 1

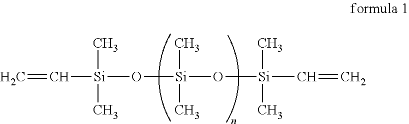

Preferably the number average weight in case this polymer is used is chosen in the range of 20 000 to 200 000 g/mol.

A curing agent as defined herein is any agent that can chemically react with the matrix pre-polymer to result in a solidification, e.g. a polymerisation reaction. Preferably the curing agent is a cross-linking agent. The curing agents can be chosen from the group of curing agents that are suitable to react with the chosen silicone (pre)-polymer.

A suitable amount of curing agent can be determined, depending upon the type of curing agent and the quantity and nature of the other components in the composition. Preferably, the curing agent is present in an amount of at least 0.1 wt. % based on the total weight of the curable composition (i.e. the mixture of components A, B and—if present—C), more preferably at least 5 wt. %. The amount of curing agent is preferably less than 15 wt. %, more preferably less than 10 wt. %.

Preferably the curing agent is present in the composition in an amount providing a number of functional groups in the range of 1-10 times the number of functional groups that is provided by the silicone (pre-)polymer.

Functional groups, as used herein, are those functional groups that are capable of participating in the curing, in particular by being capable of reacting with a functional group of another molecule (curing agent or silicone (pre-)polymer) in the composition. Examples of functional groups are vinyl groups, acryloyl groups, methacryloyl groups and hydride groups. Vinyl groups are in particular preferred in the silicone (pre-)polymer. Hydride groups are in particular preferred in the curing agent.

Examples of suitable curing agents are polyalkylhydrosiloxane polymers, including fluorinated polyalkylhydrosiloxane polymers, functionalised molecular silica compounds, such as Vinyl Q® and P.O.S.S. compounds. Very good results, in particular in combination with a silicon (pre-)polymer have been achieved with a polyalkylhydrosiloxane polymer. When used in combination with a silicon (pre-)polymer, the molar ratio of hydride to vinyl functional groups is preferably 1:1 to 10:1.

A preferred polyalkylhydrosiloxane polymer as a curing agent is a copolymer of alkylhydrosiloxane moieties and dialkylsiloxane moieties, preferably of methylhydrosiloxane moieties and dimethylsiloxane moieties.

Preferably the amount of dialkylsiloxane moieties—in particular dimethylsiloxane moieties—and/or the amount of alkylhydrosiloxane moieties—in particular methylhydrosiloxane moieties—in a polyalkylhydrosiloxane polymer is 1-100, and more preferably 5 to 20. The dialkylsiloxane-alkylhydrosiloxane copolymer may be a random, alternating or block copolymer.

Component A, component B and/or component C, may comprise a silicone base. A silicone base is a silicone oil, which may be a linear or cyclic compound, which does may be essentially free of functional groups that can participate in the curing reaction in a mixture consisting of components A and B (or components A, B and C) in the presence of the curing catalyst at 37° C. Dimethicone is an example of a silicone base.

Optionally, one or more of the components comprise a filler, which may be any physiologically acceptable filler that is compatible with the silicone polymer. Suitable fillers are preferably based on EP-A 1 435 249, in particular paragraphs, [0058]-[0067] of which the contents are incorporated by reference. Preferably, a filler selected from the group consisting of silica nanofillers, molecular silica, clay nanofillers, mica nanofillers, polymeric microfibres and glass microfibers. In particular, good results have been achieved with an amorphous silica filler, in particular an amorphous silica nanofiller.

The amount of filler in the composition depends inter alia on the type of filler and the concentration of other particulate substance(s), such as the metallic contrast agent particles. Also, a curing agent may comprise a filler component, such as vinyl Q. Also the desired characteristics of the composition after curing may play a role in determining the concentration. The skilled professional will readily be able to determine a suitable concentration. Typically, if present, the filler is present in an amount of at least 1 wt. %, based on the total weight. The upper limit is essentially determined by the amount of other constituents, present in the composition. For practical reasons, the amount of filler is usually less than 50 wt. %, based upon the total weight of the composition. Preferably the concentration is at least 2 wt. %, in particular at least 15 wt. %. The amount of filler is preferably less than about 45 wt. %, more preferably less than about 40 wt. %. in particular less than 45 wt. %. Preferably, the filler particles are nano particles, i.e. having a diameter of less than 1000 nm, in particular of 10-500 nm.

Preferred is a nano-sized silica filler, e.g. a molecular silica filler. Although such filler may contribute to radio opacity, the inventors found the use of such filler without further X-ray contrast agent not entirely satisfactory.

In an embodiment, the surface of the filler is hydrophobic, e.g. due to chemical modification. The term hydrophobic filler is used herein to describe a filler, of which the surface has been treated with a non-polar compound that dissolves better (i.e. has a higher solubility) in an organic solvent such as an alkane than in water.

An important aspect of the invention is the metallic X-ray contrast agent. The term ‘ metallic’ is used herein in the strict sense, namely to refer to the metallic form of one or more elements, unless specified or evident otherwise (e.g. when referring to metal ions or to a metal salt). In accordance with the invention it has been found unnecessary to provide the particles with a coating. Thus, the metal particles are generally uncoated. Good results have been achieved with tantalum particles. Another preferred contrast agent is tungsten. Other metal particles that may be provided in component B are gold particles, platinum particles and silver particles, of which platinum is preferred.

The inventors found that commercially available metallic contrast agents may contain a substantial amount of non-metallic material, such as carbon. E.g. the carbon content may be about 50%.

The metallic X-ray contrast agent particles in kit of parts, biocompatible composition, or cured material according to the invention, preferably at least substantially consist of a metal, in particular tantalum, more preferably for 90-100 wt. %, in particular 95-100 wt. %.

Further, the inventors found that the industrially applied measure for the particle size of metallic microparticles, suitable for use as contrast agents, the FSSS (Fisher Sub sieve Sizer), as used directly after production, does not or not necessarily reflect the particle size as determined by scanning electron microscopy (SEM, wherein the size is determined by the ‘longest enveloping circle’ of the particles, which may be agglomerated) at a later stage (shortly before its application, remote from the production facility). This may be due to the fact that the particles in practice tend to be agglomerates formed from primary particles, as initially prepared at a production facility.

The metal particles used as a contrast agent in a composition for use according to the invention usually have a particle size of about 15 μm or less, as determined by SEM (using the ‘longest enveloping circle’ of the particles, which may be agglomerates), preferably of about 10 μm or less.

Preferably, at least 90% of the total weight of the metallic X-ray contrast agent particles has a particle size of 10 micro-meter or less, as determined by SEM (using the ‘longest enveloping circle’ of the particles to determine size).

In particular, good results have been achieved with metallic X-ray contrast agent particles having a (number) average particle size of 9 micrometer or less, in particular in the range of 5-9 micrometer, as determined by SEM (using the ‘longest enveloping circle’ of the particles to determine size). Such a size has been found to be important to maintain the particles well-dispersed, also if the fluid component is exposed to an ambient temperature for some time, e.g. a few hours, in particular more than a day.

In practice, at least 50 wt. % of the particles preferably has a size of at least 200 nm, in particular of at least 500 nm, more in particular of at; least 1 micrometer, as determined by SEM (using the ‘longest enveloping circle’ of the particles to determine size).

In an advantages embodiment, the metal contrast agent particles have an irregular shape, such as a more or less flake-like or chip-like shape. Thus, the particular are preferably non-spheroidal. Of such particles, the aspect ratio (ratio longest diameter to thickness) usually is 2 or more, preferably 3 or more, in particular at least about 5. In particular at a relatively low viscosity a relatively high aspect ratio is advantageous for storage stability. The aspect ration may be up to 20 or even higher. Good results have been achieved with particles having an aspect ratio of about 10 or less.

In particular in non-agglomerated stated, the metal particles usually have a particle size of about 10 μm or less (as determined by FSSS). Non-agglomerated particles (that may form an agglomerate) or smaller particles of which an agglomerate is composed are also referred to in the art as primary particles. Preferably, at least 90% of the total weight of the metallic X-ray contrast agent primary particles has a particle size of less than 7 micro-meter, more preferably of less than 5 μm, in particular of about 3 μm or less, more in particular of about 2 μm or less, as determined by sieving (using a Fisher Sub Sive Sizer, FSSS).

It was found that a relatively low concentration of the metal particles in the curable composition provides sufficient radio opacity for monitoring the administration of the composition in vivo. The concentration in the curable composition (components A, B and—if present—C mixed together) is usually in the range of 1-10 w/v % or less, in particular in the range of 1.5-8 w/v %, preferably in the range of 2-5 w/v %. The concentration in component B is usually 1-30 w/v %, in particular 2-16 w/v %, preferably 4-10 w/v %.

Component B usually further comprises a curing catalyst, although in a specific embodiment, the curing catalyst is provided in a separate fluid component (component C). Very good results have been achieved with a platinum catalyst, in particular for curing a composition comprising a matrix pre-polymer with vinyl units as reactive site and a curing agent with hydride units as active site.

Highly preferred examples of platinum catalysts are platinum complexes, in particular platinum complexes selected from the group consisting of platinum-divinyltetramethyldisiloxane complexes. The concentration of curing catalyst can readily be determined depending upon the composition and the desired curing time. In particular good results have been achieved with a platinum catalyst in a concentration of at least 5 ppm (based upon the total weight of the biocompatible polymer composition). Particular favourable with respect to the curing time has been found to be a concentration of about 5 to 500 ppm.

In a specific embodiment, the kit of parts has a first container, containing component A and a second container containing component B in a volume to volume ratio in the range of 0.9:1 to 1:0.9, wherein component A comprises
10-85 wt. %, preferably 30-70 wt. % silicone (pre)polymer, preferably a vinyl terminated polydimethylsilicone
0.1-50 wt. %, preferably 1-25 wt. % curing agent, preferably a siloxane crosslinker.
0.5-50 wt. %, preferably 1.0-20 wt. % filler, preferably amorphous silica (nano)particles
0-50 wt. % silicone base
   and wherein component B comprises
20-75 wt. % silicone (pre)polymer, preferably a vinyl terminated polydimethylsilicone
2-10 w/v. %, preferably 3-8 w/v. % metallic contrast agent, preferably tantalum or tungsten particles
0.1-10 wt. %, preferably 1-6 wt. % curing catalyst, preferably a platinum-divinyltetramethyldisiloxane complex
0-50 wt. % silicone base.
   The mixture of components A and B in this embodiment preferably has a viscosity of 3000-10000 cSt, more preferably 5 000-8 000 cSt.

As mentioned above, the invention further relates to a method for preparing a curable silicone polymer composition, comprising mixing the components A and B, or—if component C is present—the components A, B and C of a kit of parts according to the invention. Mixing can be done in any type of mixer suitable for mixing viscous liquids. A static mixer is a convenient mixing device. Mixing is conveniently carried out a temperature of 70 C or less, in particular about 40° C. or less, preferably at a temperature in the range of 15−37° C., e.g. at a temperature in the range of 18−30° C. If a curing catalyst is also present in the composition or if the composition is exposed to suitable electromagnetic radiation, such as ultraviolet light, the composition can be cured rapidly. It is possible to fully cure the composition, typically within less than 15 min, preferably within 10 min, in particular within 5 min without needing to increase the temperature. Thus, the composition will readily cure in vivo.

In an embodiment, the kit of parts is used for in vivo vessel repair, in particular for treatment of an aneurism. A suitable procedure can be based on EP-A 1 435 249 or EP-A 2601995, of which the contents with respect to this use are enclosed by reference. Thus the present invention also relates to a method of treating a body cavity or body vessel—preferably an aneurysm in a blood—vessel, with a curable composition made with the components A and B (and optionally C) of a kit of parts according to the present invention, said method comprising the steps of covering the inner wall of the vessel or cavity with an essentially cylindrical layer of the composition and curing the composition. Obviously the curing by and large takes place after covering the inner wall, although it is possible that the curing is initiated shortly (typically up to about 1-10 min) before applying the composition to the wall.

Preferably, the composition is applied to the inner wall by using an apparatus comprising a catheter with at the distal end an expandable, essentially cylindrical carrier, which carrier is inserted in the vessel or cavity, wherein the composition is applied between the outer wall of the carrier and the inner wall of the vessel or cavity, wherein the carrier is expanded and has—in expanded state at least one, preferably two rounded shoulders, at a distance from one another, which shoulders are in contact with the cavity or vessel wall, such that a filling space for the composition is formed between the two shoulders, the outer wall of the carrier and the inner wall of the vessel or cavity, and wherein this filling space is provided with the composition. The composition can for example be applied to the filling space via one or more holes in the carrier.

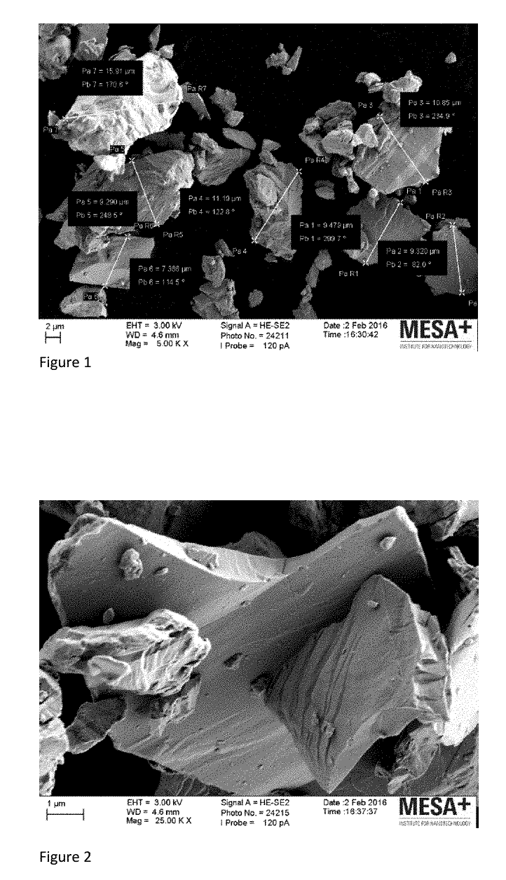

A preferred example of a carrier is a balloon that is expansible under influence of pressure, e.g. transferred via a liquid or a gas. The balloon is brought to the site to be treated, e.g. the aneurysm, where it is expanded. Via a catheter, the composition is then injected into the space between balloon and vessel wall. FIG. 1 of EP-A 1 435 249 shows an example of an aneurysm in the aorta. The arteries 1 are temporarily blocked from blood circulation with the help of three balloon catheters 2. As shown in this Figure each of the balloons has one rounded shoulder 2a. One catheter comprises an echo sounder 4 to locate the renal arteries 1a and 1b. The biocompatible polymer composition—mixed with a curing catalyst composition is injected via another catheter or a needle that has also been introduced at the aneurysm via an artery.

The invention further relates to the treatment of a bone with a curable polymer composition, using a procedure as described in EP-A 1 435 249. A human or an invertebrate in general may be effectively treated with a curable polymer composition in order to reduce the risk of complications of a future bone fracture. The bone treated in accordance with this aspect of the invention is preferably a collarbone or a hip.

Typically the (non-broken) bone is provided with a curable polymer composition. Suitable compositions are known in the art, e.g. as described herein or in one of the references cited in the present description.

In accordance with a method for treating a bone, a cavity is made in the bone, which may be done by a method generally known in the art, e.g. in a way known to introduce osteosynthetic material into a bone. Thereafter the cavity is provided with the curable composition and the composition is cured. FIG. 4 of EP-A 1 435 249 schematically shows by means of an example how a bone may be provided with the curable composition. FIG. 4A shows how a drill 2 is directed at a bone 1 and used to drill a hole (FIGS. 4B and 4C). Removal of the drill 2 provides a cavity 3 in the bone (FIG. 4D) that is filled with the curable composition 5 (FIG. 4E), which is subsequently allowed to cure. If at a later moment the bone is fractured (FIG. 4F; arrows F indicate where forces are applied), the cured composition keeps the fractured bone parts 1a and 1b in place, or at least reduces shifting of the bone parts (FIG. 4G).

The cavity is preferably provided along at least a substantial part of the bone, for instance by drilling, and filled with the curable composition, after which the composition is cured. The cured composition thus preferably forms an elastic rod-like structure.

If the bone breaks after the composition is cured, the cured composition maintains or swiftly brings back the broken parts essentially in the right position, thus avoiding or at least reducing the risk of complications due to shifting of the bone parts.

Such a method may for instance very suitably be carried out in combination with a surgery that has to be performed on a patient for an acute reason, for instance on a patient of which one of the hips or one of the collarbones has been broken.

Accordingly, the invention also relates to the use of a curable polymer compositions in the manufacture of a physiologically acceptable composition for prophylactic treatment of a bone, preferably a hip or a collarbone. Such prophylactic treatment helps to avoid or at least reduces the risk of complications after fracture.

In an embodiment, the kit of parts is used to prepare a curable composition that is used to repair an endoleak of a graft or stent-graft in an artery, in particular a type II or a type I endoleak. The repair can be done using a translumbar needle approach. By filling the trough endoleak circulated part of the aneurism with a solid mass of polymer the entry and exit branches (type 2) or in a later phase the entry spot between graft and aorta wall (type 1) and the exit vessels are sealed of. A suitable technique is described in Gorlitzer et al. Interact CardiVAscThorac Surg (2008) 7 (5) 781-784. Compared to this material, the curable composition obtained by mixing components A, B (and optionally C) offers advantages. For example, no DMSO or other organic solvent is required. DMSO can cause undesired side-effects to the patient, e.g. headaches. Further, they noticed a number of drawbacks of EVA polymer in the treatment of endoleaks. In particular, they found that during or after precipitation, (small) fragments of solidified EVA may be carried away in the bloodstream. This is a drawback both because it may weaken the repaired spot and because the fragments may form emboli in small blood vessels.

In a method wherein the curable pre-polymer composition is for use as an adjuvant filling of the aneurism in a method wherein an endo-graft is placed in the aneurism, the endo-graft can be placed by a known EVAR technique, after which the space between inner wall of the blood vessel and the outer wall of the endo-graft is filled with the composition, which then solidifies in situ.

The invention is now illustrated by a number of examples.

### EXAMPLE 1

When tantalum particles (average diameter >5 micrometer) were added to a fluid curable polymer composition (viscosity about 6000-7000 cSt) comprising vinyl terminated PDMS and a curing agent (such as polyalkylhydrosiloxane), it was observed that spontaneous curing observed. Also it was observed that the particles were not dispersed very well.

However, spontaneous curing was avoided by adding the tantalum particles to a comparable fluid composition of vinyl terminated PDMS, curing catalyst (Pt-complex) without the curing agent (component B).

In a further experiment about 10 w/v % or less of tantalum particles with 90% of the total weight having a primary particle size of less than 5 micro-meter or of about 2 micrometer or less, or of about 1 micrometer or less was added to a fluid composition of vinyl terminated PDMS, curing catalyst (Pt-complex), amorphous silica filler and silicone oil, without the curing agent (component B). After more than 3 months of storage, no substantial change in viscosity or settling of the tantalum particles was observed. This component B was mixed with a fluid composition having about the same viscosity (6000-7000 cSt) of vinyl terminated PDMS, curing agent, amorphous silica filler and silicone oil, without catalyst and without metallic contrast agent particles (component A). The resultant mixture was found to cure well at about ambient temperature, forming a homogeneous, consistent solidified, elastic mass, with good X-ray visibility.

### EXAMPLE 2

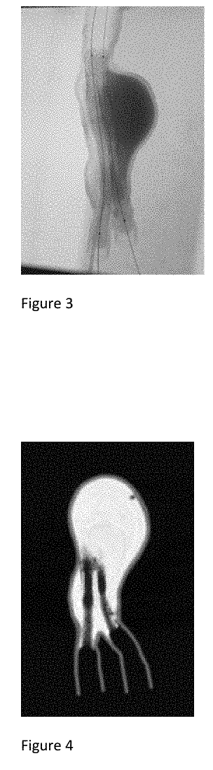

Analogous to Example 1 kits of parts was made, using tantalum particular having an average particle size (agglomerate size) of 8.6 μm, ranging from 1 μm to 10 μm. No large particles were found. The shape was very irregular, like chips (see FIGS. 1 and 2). The particles contained 95 wt % tantalum and 3.7 wt % Carbon. Using the kits of parts, curable polymer compositions were made having 2, 3, 4, 5, 6, or 7 wt. % tantalum. Best results were obtained with tantalum. Thereafter several filling tests in 3D printed patient aneurysms were carried out to verify that this % tantalum filling would not hamper the visibility of other interventional tools. It was found that the other endovascular tools like stents, wires etc. were still well visible, so the tantalum filling of the polymer is not hampering this (see also FIG. 3).

Thereafter, a check under CT-scan to verify that the cured composition comprising 5% tantalum did not give rise to excessive scattering, rendering CT-scan useless. This is of extra importance in EVAR procedures since CT-scans are the golden standard for detecting endoleaks (see also FIG. 4). As can be seen from figure x, the scattering is minimal. The amount of polymer is clearly visible, however without hampering the visibility of other endovascular tools like the endograft.

1. A kit of parts suitable for preparing a cured biocompatible silicone polymer material including a metallic X-ray contrast agent, the kit comprising two or more containers, each containing a fluid component, which components, when mixed, form a fluid curable biocompatible polymer composition, which upon curing forms the cured biocompatible silicone polymer material, wherein
a first container contains a fluid component A, which component A comprises a curable silicone (pre-)polymer and a curing agent, which curing agent is a polyalkylhydrosiloxane polymer, and which component A is essentially free of curing catalyst and metallic X-ray contrast agent particles, and
a second container contains a fluid component B, which component B is a dispersion comprising metallic X-ray contrast agent particles, and which is essentially free of curing agent.

2. The kit of parts according to claim 1, wherein the kit of parts further comprises a curing catalyst, preferably a platinum complex, which curing catalyst is present in component B or in a third container, comprising a fluid component C.

3. The kit of parts according to claim 1, wherein component B comprises a curable silicone (pre-)polymer.

4. The kit of parts according to claim 1, wherein the curing agent is a polyalkylhydrosiloxane copolymer comprising alkylhydrosiloxane moieties and dialkylsiloxane moieties, more preferably comprising methylhydrosiloxane moieties and dimethylsiloxane moieties.

5. The kit of parts according to claim 1, wherein at least one of said components contains a filler selected from the group consisting of silica nanofillers, molecular silica, clay nanofillers, mica nanofillers, polymeric microfibres and glass microfibers.

6. The kit of parts according to claim 1, wherein at least one of said components is an amorphous silica filler or an amorphous silica nanofiller.

7. The kit of part according to claim 5, wherein the filler is present in an amount of 1-50 wt. % based on the total weight of the curable polymer composition, obtained when mixing the full contents of the first container and the second container.

8. The kit of parts according to claim 1, wherein the metallic X-ray contrast agent particles comprise tantalum particles.

9. (canceled)

10. The kit of parts according to claim 1, wherein the metallic X-ray contrast agent particles are non-spheroidal, flake-shaped, or chip-shaped, or wherein at least 90% of the total weight of the metallic X-ray contrast agent particles has a particle size of 10 micro-meter or less, as determined by SEM.

11. The kit of parts according to claim 1, wherein the metallic X-ray contrast agent particles content is in the range of about 1 to about 7 w/v %, based on the total weight of the curable polymer composition, obtained when mixing the full contents of the first container and the second container.

12. The kit of parts according to claim 11, wherein the metallic X-ray contrast agent particles content is in the range of about 1 to about 5 w/v %, based on the total weight of the curable polymer composition, obtained when mixing the full contents of the first container and the second container.

13. The kit of parts according to claim 1, wherein the curable polymer composition has a viscosity at 25° C. in the range of 2 000 to 12 000 cSt at 25° C.

14. The kit of parts according to claim 1, wherein the curable polymer composition has a viscosity at 25° C. in the range of at least 100 cSt and less than 2 000 to cSt at 25° C.

15. The kit of parts according to claim 1, comprising component A and component B in a volume to volume ratio in the range of 0.9:1 to 1:0.9,
wherein component A comprises:
10-85 wt. %, preferably 30-70 wt. % silicone (pre)polymer
0.1-50 wt. %, preferably 1-25 wt. % curing agent
0.5-50 wt. %, preferably 1.0-20 wt. % filler
and wherein component B comprises:
20-75 wt. %, preferably 30-70 wt. % silicone (pre)polymer
2-10 w/v. %, preferably 3-8 w/v. % metallic contrast agent
0.1-10 wt. %, preferably 1-6 wt. % curing catalyst.

16-22. (canceled)

23. The kit of parts according to claim 1 for use in the treatment of a subject having a vascular disease.

24. The kit of parts for use according to claim 23, wherein the treatment involves in situ formation of a stent of a cured silicone polymer material in a blood vessel.

25. The kit of parts for use according to claim 23, wherein the treatment comprises the treatment of an arterial aneurism, in particular an abdominal aortic aneurism, a thoracic aortic aneurism or an aneurism in an iliac artery.

26. The kit of parts or curable polymer composition for use according to claim 23, wherein the treatment comprises a repair of an endoleak of a graft or stent-graft in an artery, in particular a type II or a type I endoleak.

27. The kit of parts according to claim 23 for use in the treatment of an aneurism, wherein the composition is for use as an adjuvant filling of the aneurism in a method wherein an endo-graft is placed in the aneurism.

28. (canceled)

29. The kit of parts according to claim 8, wherein the tantalum particles comprise at least 95 wt. % tantalum, based on the weight of the particles.
